(19) United States
(12) Patent Application Publication
(10) Pub. No.: US 2024/0398532 A1
(43) Pub. Date: Dec. 5, 2024

(54) IMPLANTS AND METHODS FOR THE FIXATION OF TISSUE

(71) Applicant: Brent Richard Collins, Long Lake, MN (US)

(72) Inventor: Brent Richard Collins, Long Lake, MN (US)

(21) Appl. No.: 18/688,302

(22) PCT Filed: Aug. 30, 2022
(86) PCT No.: PCT/US2022/041966

§ 371 Date: Feb. 29, 2024

Related U.S. Application Data
(60) Provisional application No. 63/239,153, filed on Aug. 31, 2021.

(51) Int. Cl. A61F 2/00; A61F 2/04

(52) U.S. Cl. CPC A61F 2/0022; A61F 2/04; A61F 2002/044; A61F 2250/0067; A61F 2250/0098

(57) ABSTRACT

Implants for repairing or preventing tissue migration disorders, including herniated or prolapsed tissue which has migrated from a desired or anatomically correct position in humans. The implants may include features for holding therapeutic substances near the surgical site, such as platelet rich plasma. Also described are methods of using the implants, methods of manufacturing the implants, and kits that include the implants.

### BACKGROUND

Tissue migration or relative tissue separation, such as herniation or prolapse, is a common clinical occurrence that can have sequelae ranging from reduced quality of life to significant morbidity and death.

Management of tissue migration, such as herniation and prolapse, can be complicated. Interventional techniques can be challenging, and re-migration is common and may require subsequent intervention. These difficulties challenge care providers, present risks to patients and introduce considerable cost to the healthcare system.

Current therapeutic approaches and associated technologies have limitations that contribute to these complications.

### SUMMARY

The present invention is directed towards implants and methods relating to the re-approximation, fixation and/or prevention of migration of tissue, including, but not limited to, herniated or prolapsed tissue which has migrated from a desirable or anatomically correct position in humans or other mammals.

In some embodiments, the present invention includes a surgical implant for preventing or treating tissue prolapse or herniation disorders in a human patient. The implant includes a tubular portion and a flange portion. The tubular portion includes a first end and a second end and defines a lumen. The lumen extends from the first end to the second end of the tubular portion, and the tubular portion is configured to allow tissue of the patient to be positioned within the lumen. The flange portion includes a first surface and a second surface and extends radially about a circumference of the tubular portion. One or both of the first surface and the second surface is configured to abut tissue of the patient.

In some embodiments, the present invention includes a method of preventing or treating tissue prolapse disorders in a human patient. The method includes providing a surgical implant that includes a tubular portion, wherein the tubular portion includes a first end and a second end and wherein the tubular portion defines a lumen extending from the first end to the second end of the tubular portion. The tubular portion is configured to allow tissue of the patient to be positioned within the lumen. The surgical implant also includes a flange portion. The flange portion includes a first surface and a second surface. The flange portion extends radially about a circumference of the tubular portion. One or both of the first surface and the second surface is configured to abut tissue of the patient. The method further includes positioning tissue of the patient within the lumen of the tubular portion and positioning the flange portion of the implant against tissue of the patient.

The present invention also includes a surgical implant for preventing or treating tissue migration, the surgical implant comprising a first layer and a second layer, wherein the first layer is a structural layer configured to support tissue after implantation into the anatomy of a patient and wherein the second layer is an absorbent layer configured to absorb and hold a therapeutic agent (e.g., platelet rich plasma) in a desired position within the anatomy of the patient.

The present invention also includes surgical kits, wherein the kits include at least one of the inventive implants described herein.

The present invention also includes methods of manufacturing the inventive implants.

### BRIEF DESCRIPTION OF THE DRAWINGS

In the drawings, which are not necessarily, drawn to scale, like numerals may describe similar components in different views. The drawings illustrate generally, by way of example, but not by way of limitation, various embodiments discussed herein.

FIGS. 1A-1C shown three schematics of the upper digestive tract of the human body.

FIGS. 2A-2H illustrate various perspective views of surgical implants of the present invention.

FIGS. 3A-3C illustrate schematics of a portion of the upper digestive tract of the human body after implants of the present invention have been placed into a desired position.

FIGS. 4A-4C illustrate assembled and exploded views of an implant of the present invention.

FIG. 5 illustrates an implant of the present invention that includes a “scarf” design.

FIG. 6 illustrates an implant of the present invention that includes a pad of absorbable material encompassing a matrix of non-absorbable material.

FIG. 7 illustrates an implant of the present invention that includes a non-absorbable material adjacent and external to an absorbable material.

FIG. 8 illustrates an implant of the present invention that includes an absorbable first material that does not encompass or envelope a second material.

FIG. 9 illustrates an implant of the present invention that includes “outshoots” or extensions that scar tissue will form around.

FIG. 10 illustrates an implant of the present invention that includes a series of contours that undulate along a major longitudinal axis of a strip of material.

FIG. 11 illustrates an implant of the present invention that includes a slot or aperture used to facilitate attachment of the implant to itself or surrounding tissues.

FIG. 12 illustrates an implant of the present invention that includes a plurality of pockets formed within a strip of material.

### DETAILED DESCRIPTION

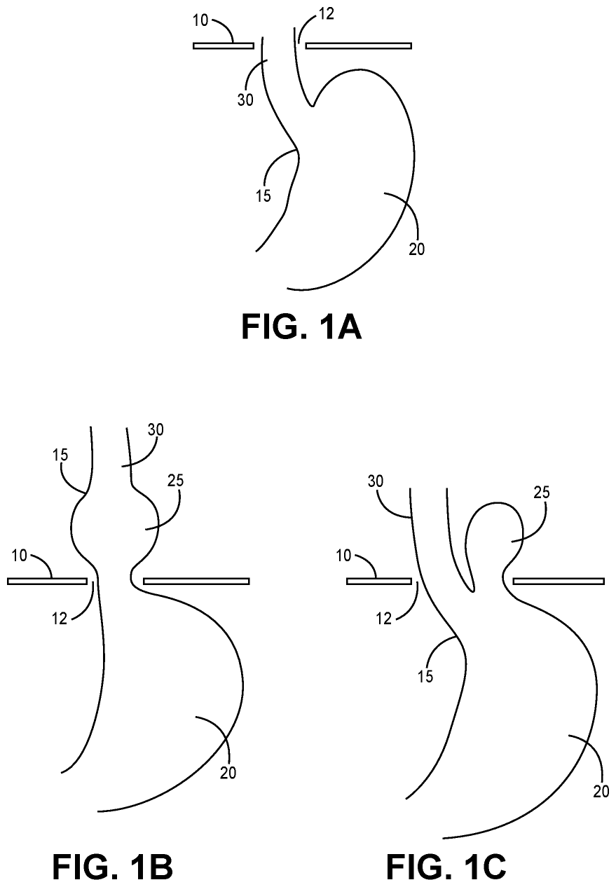

In some embodiments, the invention includes implants for the treatment of hiatal hernias. FIGS. 1A-1C show three simplified schematics of the upper digestive tract of a human body, two of which show hiatal hernia disease states.

FIG. 1A shows a view of normal human anatomy (i.e., non-diseased), with esophagus 30 extending through esophageal hiatus 12 defined by diaphragm 10. The distal end of esophagus 30 joins with a proximal portion of stomach 20 at the lower esophageal sphincter 15 below or inferior to hiatus 12 and diaphragm 10.

FIG. 1B shows a view of human anatomy afflicted with a “sliding” hiatal hernia where a distal portion of the esophagus 30 and the esophageal sphincter 15 have moved superior to hiatus 12 and diaphragm 10. Herniated portion 25 of stomach 20 extends superior of diaphragm 10 as well.

FIG. 1C shows another view of human anatomy afflicted with paraesophageal or “rolling” hiatal hernia where a distal portion of esophagus 30 and the esophageal sphincter 15 are mostly inferior to hiatus 12 and diaphragm 10 but herniated portion 25 of stomach 20 extends superior of diaphragm 10.

Some embodiments of the present inventive implants permit a surgeon to reliably treat hiatal hernias and reflux diseases while simultaneously treating the defects in the esophageal hiatus often associated with poor outcomes. For example, implants of the present invention may provide support to the hiatus and lower esophageal sphincter while acting as a reservoir for humoral factors (e.g., platelet rich plasma or “PRP”) that have been shown to facilitate or accelerate formation of dense connective tissue in or around the esophageal sphincter and/or the crus muscles.

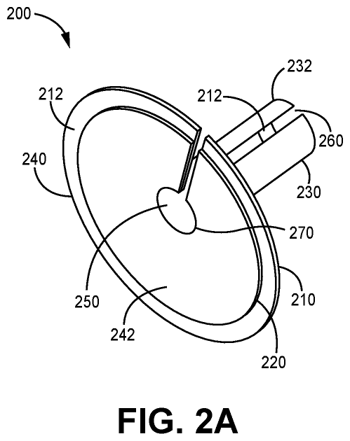

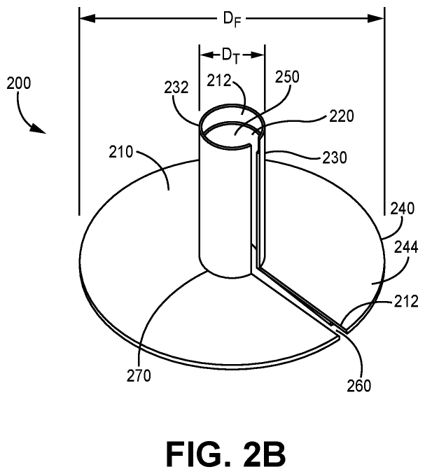

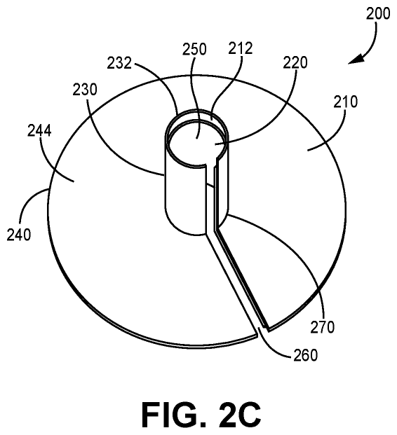

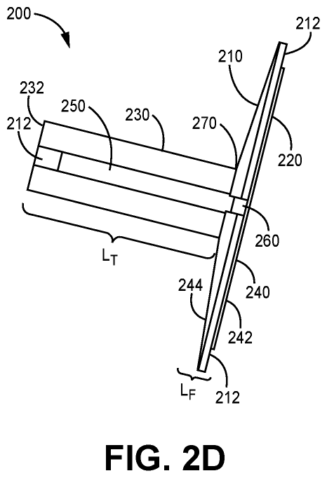

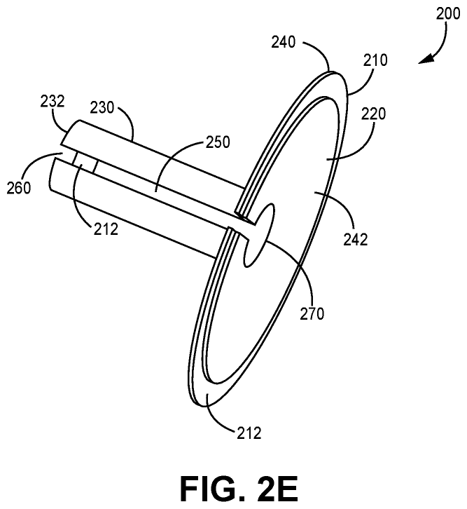

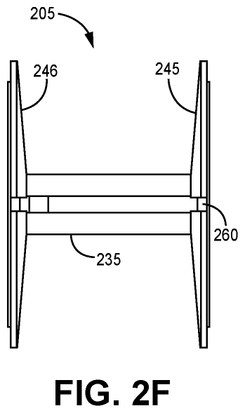

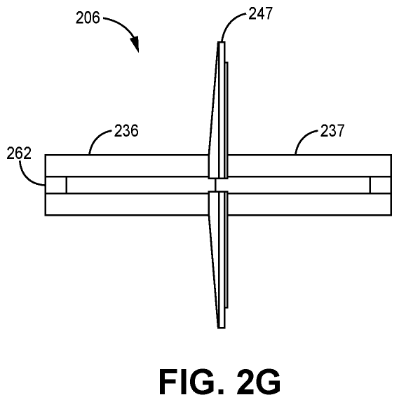

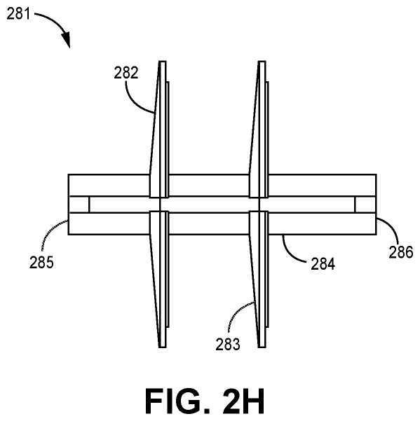

FIGS. 2A-2E illustrate various perspective views of one embodiment of the invention in the form of surgical implant 200, which is useful for treating hiatal hernias, prolapse, or other types of tissue migration maladies. Implant 200 includes first layer 210 and second layer 220. First layer 210 and second layer 220 are disposed adjacent to each other. Implant 200 includes tubular portion 230 and flange portion 240.

FIG. 2A illustrates a perspective view of implant 200 showing surface 242 of flange portion 240 and end 232 of tubular portion 230. Tubular portion 230 defines lumen 250. Installation slit 260 runs along the longitudinal length of tubular portion 230 and extends radially through flange portion 240. First layer 210 is slightly larger than second layer 220, resulting in overhang areas 212 near the outer circumference portion of flange portion 240 and end 232 of tubular portion 230. Tubular portion 230 is joined to flange portion 240 at joint 270. While first layer 210 is shown as slightly larger than second layer 220 in the embodiment of the invention illustrated in FIGS. 2A-2E, in other embodiments of the invention the first layer is the same size as the second layer or the first layer is smaller than the second layer.

FIGS. 2B and 2C illustrate perspective views of implant 200 showing surface 244 of flange portion 240 and tubular portion 230. Joint 270 secures tubular portion 230 with flange portion 240, with tubular portion 230 extending from flange portion 240 at roughly a 90° angle. While flange portion 240 extends at roughly a 90° angle from tubular portion 230, in other embodiments of the invention, the one or more flange portions can extend from the tubular portion at different angles, such as, for example, at an angle of about 5°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, or at any angle or range of angles that fall on or between any of the angles specifically mentioned here. FIG. 2B illustrates that tubular portion 230 has diameter D_{T} and flange portion 240 has diameter D_{W}. In some embodiments, the diameter of a tubular portion is sufficient to reduce tension placed on the esophagus and/or crurual closure with enough resistance to restore competence to the lower esophageal sphincter but without causing tissue erosion or dysphagia after implantation. As can be seen in FIGS. 2B and 2C, second layer 220 is disposed adjacent to first layer 201 in tubular portion 230.

In some embodiments, a suture is used to join tubular portion 230 with flange portion 240 at joint 270. The suture could be made of a material that is one or more of radiopaque, non-radiopaque, absorbable or biodegradable, and non-absorbable. In some embodiments, joint 270 includes a radiopaque material (e.g., the radiopaque suture mentioned above) so that the location of joint 270 will be visible under CT or other forms of x-ray imaging or ultrasonic imaging.

FIG. 2D illustrates a perspective side view of implant 200 showing that tubular portion 230 has a longitudinal length L_{T} while flange portion 240 is relatively short along its axial length L_{W}.

While the various parts of implant 200 can be formed with any desired dimensions for a given application, in some embodiments i) the diameter D_{T} of tubular portion 230 may be less than 10 mm, 10 mm, 11 mm, 12 mm, 13 mm, 14 mm, 15 mm, 16 mm, 17 mm, 18 mm, 19 mm, 20 mm, or more than 20 mm; ii) the diameter D_{W} of flange portion 240 may be less than 3 cm, 4 cm, 5 cm, 6 cm, 7 cm, 8 cm, 9 cm, 10 cm, or more than 10 cm; and/or iii) the length of implant 200 (which would be the sum of dimensions L_{T} and L_{W}) may be less than 2 cm, 2 cm, 3 cm, 4 cm, 5 cm, 6 cm, 7 cm, 8 cm, 9 cm, 10 cm, or more than 10 cm.

FIG. 2E illustrates a perspective view of implant 200 showing surface 242 of flange portion 240. Installation slit 260 runs radially through flange portion 240 and along the length of tubular portion 230, thereby providing access to lumen 250. For example, when implanting implant 200 into the anatomy, a user can direct tissue (e.g., esophageal tissue) through installation slit 260 so that the tissue is arranged or positioned within lumen 250.

While implant 200 shown in FIGS. 2A-2E include two layers of material disposed against one another (i.e., first layer 210 and second layer 220), some embodiments of the present invention include only one layer or more than 2 layers. For example, some embodiments of the present invention include 1, 2, 3, 4, 5, 6, 7, 8, 9, 10, 11, or 12 layers. The one or more layers of the inventive implant may be the same size and shape or one or more of the layers may have a different size or shape such that different portions of the implant have dissimilar number of layers. For example, an implant of the present invention may have a tubular portion that includes two layers while the flange portion includes only a single layer.

The layers of the present inventive implants may be formed from a wide variety of materials. For example, one or more of the layers of the inventive implant may include or be formed from a polymer that will not substantially degrade when implanted within a mammalian body (e.g., polypropylene). One or more of the layers of the inventive implant may include or be formed from a biodegradable material (i.e., a material that will degrade or be absorbed when implanted in a mammalian body over a relatively short time frame, such as 30, 60, 90, 180, or 270 days), such as polylactic acid (PLA), polyglycolic acid (PGA; e.g., Vicryl™ (polyglactin 910) available from Johnson & Johnson of New Brunswick, New Jersey), combinations of polyglycolic acid and trimethylene carbonate (e.g., Bio-ATM available from W.L. Gore & Associates, Inc. of Newark, Delaware), polycaprolactone (PCL), poly-4-hydroxybutyrate-based materials, tyrosine-derived polycarbonates, collagen-based materials (e.g., human and porcine dermis, bovine pericardium, bladder mucosa, and/or small intestinal submucosa), or combinations thereof. One or more of the layers of the inventive implant may include or be formed of a material that absorbs or retains fluids (e.g., platelet rich plasma), such as polyoxamer hydrogels (e.g., Pluronic™ F-127, available from Millipore Sigma of Burlington, Massachusetts). One or more layers of the inventive implant may include or be formed from a material that will impart resistance to the adhesion of tissue when implanted into a mammal, such as hyaluronic acid (HA), carboxymethyl cellulose (CMC), omega-3 fatty acids, or combinations thereof. One or more layers of the inventive implant may include or be formed from a material that will induce or promote the formation of scar tissue when implanted into a human or other mammal, thereby promoting in-growth of scar tissue into the implant to better secure the implant at the desired anatomical location.

The layers of the present invention can take a variety of different forms. The implant, for example, can have one or more layers that are formed into a porous mesh. Alternatively, or in addition, one or more layers of the inventive implant are in the form of a solid laminar sheet, a sheet of woven material, a sheet or coating formed by spray-coating, a sheet or coating formed by electro-spinning, a sheet or coating formed by dip-coating, a sheet or coating made by flow coating, a sheet or coating made by roll coating, a sheet or coating made by an extrusion process, or combinations thereof.

In some embodiments, one or more layers of the present inventive implant may include a pharmacologic agent or other biologically active material, such as platelet rich plasma (PRP), a clotting factor, a clotting cofactor, or combinations thereof. For example, one or more layers of the inventive implant may include activants such as thrombin, calcium dichloride, or combinations thereof. In a further example, an implant of the present invention may include a layer of a material that absorbs or retains fluids and has been impregnated with thrombin and/or calcium dichloride. A surgeon implanting the implant into a patient can optionally inject PRP into the implant or soak the implant with PRP, thereby contacting the PRP with the thrombin and/or calcium dichloride included in the layer of absorbent material and facilitating coagulation of the PRP. The absorbent material of the inventive implant can retain the PRP and localize the PRP to a portion of the anatomy of interest. In this manner, the implant of the present invention can utilize PRP to promote healing of tissue and/or tissue growth in a patient.

One embodiment of the present invention includes an implant that has a first layer made of a sheet of polyglactin or polyglycolic acid and a second layer made of type I collagen. The first layer has a pore size of about 3 millimeters, is multifilament in construction, has a weight of about 30 grams per square meter, a tensile strength of at least 16 N/cm. In other embodiments of the invention, the first layer has a pore size of less than 3 millimeters in size (e.g., 2.5 mm, 2 mm, 1.5 mm, 1 mm, 0.5 mm, 0.1 mm, 0.01 mm, 0.001 mm, 500 nm, 50 nm, 5 nm, or any size intermediate of the sizes specified herein). When implanted into a human body, the first layer of the implant should be fully absorbed in about 60 to 90 days. The second layer of type I collagen is a non-crosslinked sheet having a thickness of between 0.2 and 0.5 millimeters. When implanted into a human body, the second layer of the implant should be fully absorbed in about 60 to 90 days. The first layer is cut and/or rolled to a desired size and shape. Optionally, the first layer is heat formed or shaped. The second layer is similarly cut and/or formed to a desired size and shape. The second layer is impregnated with a therapeutic agent, such as PRP, thrombin and/or calcium dichloride. For example, the second layer can be impregnated with an agent during manufacture of the implant by solution deposition, crystallization, powder deposition, or other deposition processes as are known in the art or during use by the surgeon injecting or otherwise introducing the agent into or on the second layer. The first and second layer are joined to form the finished implant. The layers can be joined by lamination, adhesive, sewing, roll forming, or other methods as are known in the art. When implanted into a human body, the first layer may act as a “structural layer” by providing support to the overall implant and surrounding tissue. The second layer can be injected or soaked with a patient's PRP shortly before implantation. The thrombin and/or calcium dichloride of the second layer will facilitate clotting of the PRP, thereby helping to localize the PRP in or on the second layer. After implantation, the second layer will retain and localize the PRP in a desired part of the anatomy and provide for relatively slow release of platelet factors and other factors that will promote tissue healing and/or growth in the hours, days, or weeks after implantation.

While implant 200 includes a single flange portion attached to one end of a tubular portion, in some embodiments the implant of the present invention includes two flange portions with one flange portion attached at each end of the tubular portion. FIG. 2F illustrates such an embodiment in the form of implant 205. Implant 205 includes tubular portion 235, flange portion 245, and flange portion 246. Flange portion 246 is secured to one end of tubular portion 235, while flange portion 245 is secured to the opposite end of tubular portion 235. Implant 205 includes installation slit 261, which runs along the longitudinal length of tubular portion 235 and across the width of both flange portions 245 and 246. While implant 205 illustrates an embodiment where the implant has two flange portions 245, 246 positioned at opposite ends of the tubular portion 235, in other embodiments of the invention, one or both of the flange portions can be positioned points that are between the ends of a tubular portion. FIG. 2H, for example, illustrates implant 281 which includes first flange portion 282 and second flange portion 283, each positioned at a location along tubular portion 284 that is intermediate of first end 285 and second end 286. Implant 281 can be positioned within the anatomy of a patient such that the patient's diaphragm is positioned between flange portions 282 and 283.

While implant 200 and implant 205 both illustrate embodiments of the invention where a flange portion is attached to the end of a tubular portion, in still further embodiments of the invention the implants include a flange portion that is positioned at some midpoint along the tubular portion between the ends. FIG. 2G illustrates such an embodiment in the form of implant 206. Implant 206 includes flange portion 247 positioned approximately half-way along the length of the tubular portion, thereby creating a first tubular portion 236 extending from one surface of flange portion 247 and a second tubular portion 237 extending from the opposite surface of flange portion 247. Implant 206 includes installation slit 262 which extends along the entire lengths of first and second tubular portions 236 and 237 as well as through flange portion 247.

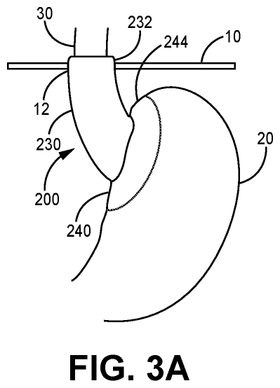

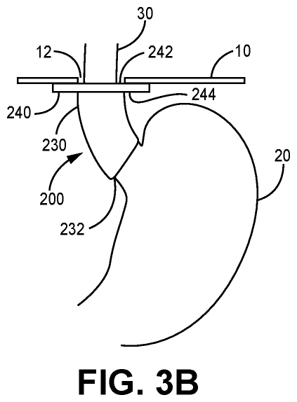

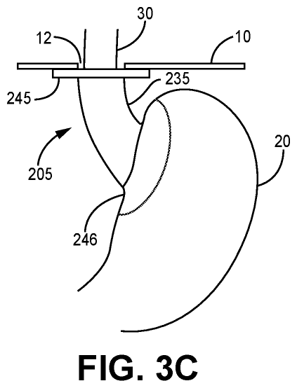

FIG. 3A shows a simplified schematic of a portion of the upper digestive tract of a human body after implant 200 of the present invention has been placed into a desired position. Esophagus 30 has been passed through installation slit 260 (not shown in FIG. 3A) of implant 200 and is now positioned along the length of lumen 250. The proximal portion of esophagus 30 extends from and is superior to end 232 of implant 200. Implant 200 is positioned such that surface 242 of flange portion 240 abuts stomach 20 and surface 244 faces away from stomach 20. Tubular portion 230, along with esophagus 30, extend through esophageal hiatus 12 of diaphragm 10.

FIG. 3B shows a simplified schematic of a portion of the upper digestive tract of a human body after implant 200 of the present invention has been placed into a desired position. In this embodiment, implant 200 has been positioned such that surface 242 of flange portion 240 abuts against diaphragm 10, while surface 244 faces away from diaphragm 10. Esophagus 30 has been passed through installation slit 260 (not shown in FIG. 3B) of implant 200 and is now positioned along the length of lumen 250. The proximal portion of esophagus 30 extends from and is superior to flange 240 of implant 200. Esophagus 30 extends through esophageal hiatus 12 of diaphragm 10, through lumen 250 of implant 200, and out end 232 of implant 200. End 232 of implant 200 is proximate to or abuts against stomach 20. Alternatively, so portion of stomach 20 could be positioned within lumen 250. For example, the lower esophageal sphincter could be positioned within lumen 250 and/or a portion of the upper stomach tissue could be positioned within lumen 250.

FIG. 3C shows a simplified schematic of a portion of the upper digestive tract of a human body after implant 205 of the present invention has been placed into a desired position. Unlike implant 200, implant 205 has two flange portions 245 and 246 secured at either end of tubular portion 235. As illustrated in FIG. 3C, implant 205 has been positioned such that flange portion 245 abuts against diaphragm 10 while flange portion 246 abuts against stomach 20. Esophagus 30 extends through esophageal hiatus 12 of diaphragm 10, through flange portion 245, tubular portion 235, and flange portion 246. In some embodiments, one or both of the flange portions are i) angled relative to the tubular portion, ii) have a concave or convex face, and/or iii) include pleating, all so as to improve the fit of the implant to the anatomy of interest in a patient.

While FIGS. 1A-3C illustrate embodiments of the invention where the inventive implant is used to treat herniation or prolapse of esophageal and stomach tissue, the inventive implants can be used to treat prolapse or herniation disorders in other parts of the body. For example, in some embodiments, the inventive implants are used to treat herniation or prolapse of rectal or vaginal tissue. For example, a portion of a rectum or sigmoid colon could be positioned within a tubular portion of an inventive implant while the flange portion could be positioned to abut against the tissue of the pelvic floor. In further examples, some or all of the tissue from a body sphincter could be positioned within a tubular portion (e.g., some or all of a lower esophageal sphincter or the rectal sphincter could be positioned within the tubular portion).

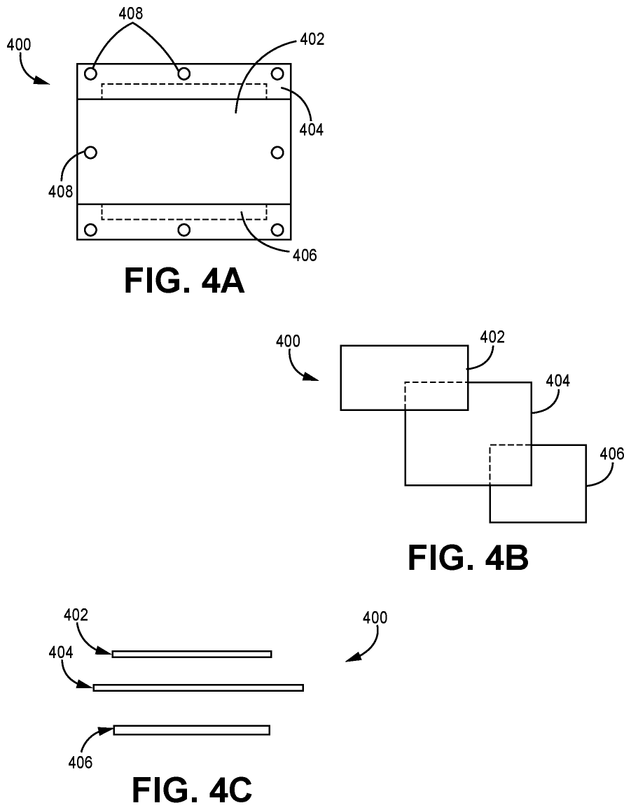

In some embodiments, the implants of the present invention include a layer that acts as a “shielding” layer to prevent adhesion to select surrounding material as other layers of the implant are absorbed. FIGS. 4A-4C illustrate one embodiment of the present invention in the form of implant 400 which includes a “shielding” layer. Implant 400 includes first layer 402, second layer 404 and third layer 406. FIG. 4A illustrates all three layers as arranged and assembled as implant 402, while FIG. 4B illustrates a perspective exploded view of implant 400 and FIG. 4C illustrates an exploded side view of implant 400. As best illustrated in FIGS. 4B and 4C, first layer 402 is arranged such that it is disposed over one side of second layer 404 while third layer 406 is disposed over the opposite side of second layer 404. As best shown in FIG. 4A, implant 400 includes points 408 arranged about the periphery or fringe of implant 400. Some or all of points 408 may be, for example, tissue anchors (e.g., stiches, staples, adhesive, etc.) for securing implant 400 to the anatomy of a patient.

Second layer 404 is a “structural layer” in that one of its purposes is to provide implant 400 with a desired amount of rigidity for a given application so that implant 400 can be held in the anatomical location of interest. First layer 402 is a “shielding layer” in that one of its purposes is to prevent patient tissue from adhering to portions of implant 400 as implant 400 is absorbed in the patient's body. Third layer 406 is an “absorptive layer” in that one of its purposes is to absorb PRP or other therapeutic components and hold the PRP or therapeutic components in a desired location of the anatomy after implantation of implant 400 so that the PRP or therapeutic components can be released over time and facilitate tissue healing and/or growth.

The present invention includes methods of manufacturing an implant. In one embodiment, the present invention includes a method of manufacturing an inventive implant that comprises, consists essentially of, or consists of:
i) Cutting a first layer material to a desired size to produce a first layer. For example, the first layer material may be a layer of 50:50 PLA/PGA copolymer which is laser cut to the desired shape.
ii) Optionally cutting additional layers of material to desires sizes and shapes and disposing them on the first layer.
iii) Molding the first layer (and any additional layers that may have been applied to the first layer) to a desired shape to produce a shaped first layer. For example, the first layer may be thermo-formed to a desired shape (e.g., a shape of a tubular portion and/or a flange portion).
iv) Optionally applying one or more additional layers to the shaped first layer. For example, a layer that is to serve as a shielding layer and/or a layer that is to serve as an absorbent layer may be applied by a suitable method to the shaped first layer (e.g., adhered via adhesive, spray coated, lamination, or sewing). In some embodiments, a layer that is to serve as an absorbent layer (e.g., type I fibrillar collagen sheet) is applied wet to a shaped first layer.
v) Packaging and labelling the shaped first layer (including any of the optional additional layers that may have been applied) to produce a packaged implant. For example, the shaped first layer may be packaged in a double-barrier tray with Tyvek™ lid.
vi) Sterilizing the packaged implant to produce a sterilized packaged implant. For example, the packaged implant could be sterilized by ethylene oxide sterilization, e-beam sterilization, or gamma irradiation sterilization.

In some embodiments, the present invention includes a method of manufacturing an inventive implant that comprises, consists essentially of, or consists of:
i) Cutting a first layer material to a desired size to produce a first layer. For example, the first layer material may be a layer of 50:50 PLA/PGA copolymer which is laser cut to the desired shape.
ii) Molding the first layer to a desired shape to produce a shaped first layer. For example, the first layer may be thermo-formed to a desired shape (e.g., a shape of a tubular portion and/or a flange portion).
iii) Applying a second layer in the form of a shield layer to the shaped first layer to produce a shielded shaped first layer. For example, a shield layer could be applied by spray coating the shaped first layer or by sewing a shield layer to the shaped first layer. In some embodiments of the inventive method of manufacture, the shield layer is applied to the first layer prior to molding of the first layer.
iv) Applying a third layer in the form of an absorptive layer (e.g., type I fibrillar collagen sheet) to the shielded shaped first layer to produce the final implant. For example, the absorptive may be applied while wet to the shielded shaped first layer.
v) Packaging and labelling the final implant to produce a packaged implant. For example, the implant may be packaged in a double-barrier tray with Tyvek™ lid.
vi) Sterilizing the packaged implant to produce a sterilized packaged implant. For example, the packaged implant could be sterilized by ethylene oxide sterilization, e-beam sterilization, or gamma irradiation sterilization.

In some embodiments, the present invention comprises, consists essentially of, or consists of one or more of the inventive implants. Optionally, the kit includes fixation devices and/or instruments for delivering the implant to a desired anatomical location within a mammal.

In some embodiments, the inventive implant comprises, consists essentially of, or consists of two types of material: i) a first material that will induce the formation of scar tissue when in contact with native tissue and ii) a second material for providing support to scar tissue induced by the first material. In some embodiments, the first material includes absorbable surgical mesh or other materials while the second material is a non-absorbable, compliant material in the form of a uniform or woven band. In further embodiments, the second material includes materials that are dynamic, non-absorbable and well tolerated by the body. In other embodiments, the inventive device comprises, consists essentially of, or consists of the first material and the first material induces the formation of scar tissue when in contact with native tissue. In still further embodiments, the first material and/or second material includes a material that can absorb or retain PRP.

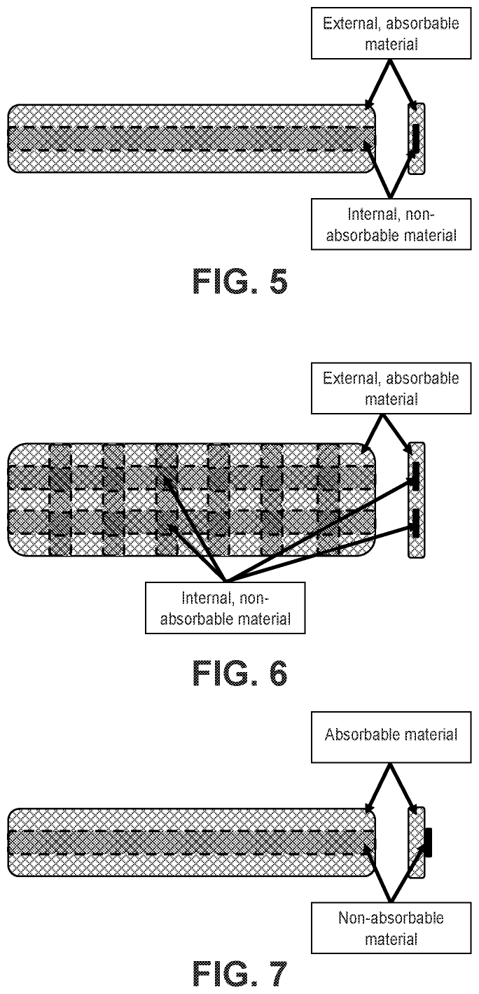

In some embodiments of the present invention, the first material encompasses or envelopes the second material. FIG. 5 illustrates an example of such an embodiment that includes a “scarf” design where a strip of the first material (referred to in FIG. 5 as the “External, absorbable material”) encompasses or envelops an inner strip of the second material (referred to in FIG. 5 as the “Internal, non-absorbable material”). The strips of the first material and the second material may have, as shown in FIG. 5, a generally rectangular prismatic shape. However, in alternative embodiments, the strips of first material and second material may be made in other shapes or formed to have other shapes as desired for a given application. For example, the strips may be formed as a cylinder and have a cross-sectional shape that is circular or forms an oval. Further or alternatively, the strips may have rounded edges or corners. This “scarf” example of the invention shown in FIG. 5 is therefore a rectangular portion of first material encompassing and running coaxially to the second material. This scarf can be placed circumferentially around reapproximated tissue that has previously migrated or demonstrated relative separation (e.g., in herniation or prolapse) such that the absorbable first material would contact surrounding native tissue and induce the formation of scar tissue thereby fixing the tissues together and preventing remigration in the future. The non-absorbable second material will become encapsulated by the newly formed scar tissue and remain in place to provide support to the scar tissue. The second material will also serve as a buttress that would prevent the internal tissue from migrating through or separating from the surrounding tissue.

FIG. 6 illustrates another example of such an embodiment that includes a pad with an absorbable first material (referred to in FIG. 6 as “External, absorbable material) encompassing a matrix of a non-absorbable second material (referred to in FIG. 6 as “Internal, non-absorbable material”). The matrix of second material may be a single unibody mesh of second material or the matrix may be formed by arranging separate strips of second material as a matrix, where the separate strips are adhered together (e.g., by heat welding or adhesives) or held in place by the first material. When the inventive pad shown in FIG. 6 is placed across tissue which has been reapproximated following migration or relative separation, the absorbable first material would contact surrounding native tissue and induce the formation of scar tissue that would fix the tissues together thereby preventing remigration; the non-absorbable second material would be encapsulated by this newly formed scar tissue and remain in place to support the scar tissue and to serve as a buttress that would prevent the internal tissue migrating or separating from the surrounding tissue. In some embodiments, the second material is made of a substance that is absorbable (e.g., a material that absorbs into the body over a time frame longer than the time frame needed to absorb the first material.

In other embodiments of the present invention, the first material does not encompass or envelope the second material or only partially encompasses or envelopes the second material. FIG. 7 illustrates such an embodiment in the form of a scarf that includes a non-absorbable second material (referred to in FIG. 7 as “Non-absorbable material”) adjacent and external to an absorbable first material (referred to in FIG. 7 as “Absorbable material). The second material can be laminated, glued or otherwise adhered to the first material. The scarf shown in FIG. 7 can be placed circumferentially around reapproximated tissue that has previously migrated or demonstrated relative separation, such as in herniation or prolapse, the absorbable first material would contact surrounding native tissue and induce the formation of scar tissue that would fix the tissues together preventing remigration; the non-absorbable second material would be encapsulated by this newly formed scar tissue and remain in place to support the scar tissue and to serve as a buttress that would prevent the internal tissue migrating through or separating from the surrounding tissue.

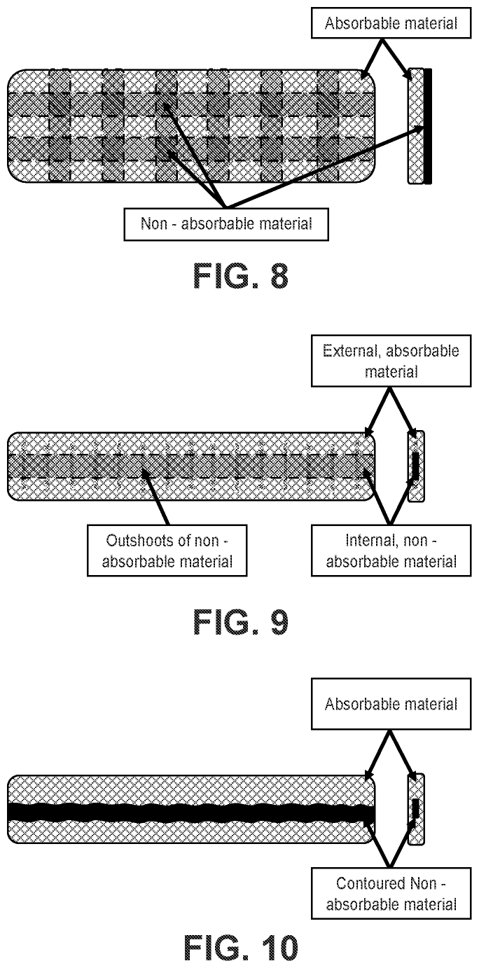

FIG. 8 illustrates another example of the present invention where a first material does not encompass or envelope the second material. FIG. 8 illustrates an example of an inventive pad that includes an absorbable first material (referred to in FIG. 8 as “Absorbable material”) arranged, layered, or otherwise approximated next to a matrix of non-absorbable second material (referred to in FIG. 8 as “Non-absorbable material”). The matrix of second material may be a single unibody mesh of second material or the matrix may be formed by arranging separate strips of second material as a matrix and then adhering the strips together and/or with the first material (e.g., by heat welding, adhesives, or stitching). When placed across tissue which has been reapproximated following migration or relative separation, the absorbable first material of the inventive pad would contact surrounding native tissue and induce the formation of scar tissue that would fix the tissues together preventing remigration; the non-absorbable second material would be encapsulated by newly formed scar tissue and remain in place to support the scar tissue and to serve as a buttress that would prevent the internal tissue migrating or separating from the surrounding tissue.

In all of these embodiments or in further embodiments, the non-absorbable second material may include “outshoots” or extensions that scar tissue would form around, further securing the relative position of the second material against the scar tissue. FIG. 9 illustrates an example of the invention where the second material includes these outshoots (referred to in FIG. 9 as “Outshoots of non-absorbable material”).

In all of these embodiments or in further embodiments, the non-absorbable second material may include features such as contours or weaves that allow compliance with physiologic functions. FIG. 10 illustrates an example of the present invention where the second material includes a series of contours that undulate along the major longitudinal axis of a strip of second material (referred to in FIG. 10 as “Contoured Non-absorbable material”).

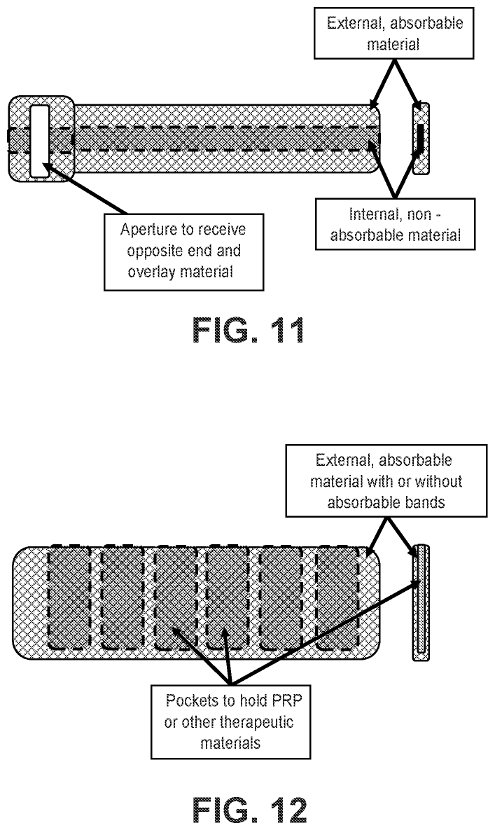

In all of these embodiments or in further embodiments, the inventive devices may include various mechanisms used to facilitate connection of the device to itself or to surrounding tissues. For example, the mechanisms used to facilitate connection may include tacks, sutures, staples or clasps as well as slots in the materials that allow one end to pass through another creating an overlap of the material. FIG. 11 illustrates one such mechanism in the form of a slot or aperture at a first end of a scarf shaped implant similar to the example shown in FIG. 5. This scarf implant can be placed circumferentially around reapproximated tissue that has previously migrated or demonstrated relative separation (e.g., in herniation or prolapse) such that the absorbable first material would contact surrounding native tissue and induce the formation of scar tissue thereby fixing the tissues together and preventing remigration in the future. The second end of the scarf implant (the end on the right of the illustration of FIG. 11) can be passed through the slot of the first end of the scarf, thereby arranging the implant such that the first and second ends overlap one another to a desired extent. Optionally, a surgeon may secure the overlapping portions to one another using sutures, staples or tacks.

The present invention also includes methods of using the implants described herein to re-approximate, fix or prevent remigration of tissue. The methods include accessing a portion of the anatomy experiencing tissue migration (e.g., herniated esophageal tissue), arranging or otherwise repositioning the migrated tissue to a desired position in the anatomy, and securing an implant of the present invention around the re-positioned tissue. In some embodiments, the inventive implants can be inserted into the body via the usual surgical approaches. For example, an inventive implant can be grasped or compressed with a surgical forceps and passed through a trocar to reach the desired implant site.

As mentioned above, the present invention includes embodiments where an implant may be used in conjunction with a therapeutic substance, such as a drug or platelet rich plasma (herein, “PRP”). One or both of the first and second materials may be configured to hold or take up the therapeutic substance, much like a sponge takes up water. A physician could, for example, soak the inventive devices in a fluidic therapeutic substance or inject such a therapeutic substance into one or both of the first or second materials either prior to, during, or after implantation of the device into a patient.

Alternatively, or in addition, the device may include features that facilitate its use with the therapeutic substance. For example, one or both of the first or second materials can be configured to define a void or “pocket” into which a therapeutic substance is injected before, during, or after implantation of the device. The pocket may include additional features to facilitate delivery of the therapeutic substance. For example, the pocket may include an absorbable sponge-like material or may include an absorbable liner barrier that would store and hold the therapeutic substance in place until after implantation in the patient. The physician can inject the therapeutic substance into the sponge material of the pocket or through the liner barrier, after which the sponge and/or liner barrier will hold the therapeutic substance within the pocket until some point after implantation of the device. FIG. 12 illustrates one embodiment of the invention that includes a plurality of pockets formed within a strip of first material. The plurality of pockets are configured to hold PRP or other therapeutic materials. As illustrated in FIG. 12, the embodiment of the inventive device does not include a second non-absorbable material embedded or encompassed within the first material. However, in some embodiments, the inventive device includes both a first material defining one or more pockets configured to hold PRP or other therapeutic materials and a second material that is non-absorable and is embedded within the first material.

Various examples have been described. These and other examples are within the scope of the following claims. The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto.

1. A surgical implant for preventing or treating tissue prolapse or herniation disorders in a human patient, the implant comprising:
a tubular portion comprising a layered composite extending axially from a distal end to a proximal end and circumferentially from a first edge to a second edge, wherein the first edge and the second edge define a first portion of an installation slit extending along a longitudinal length of the tubular portion, wherein the tubular portion defines a lumen extending from the distal end to the proximal end and configured to receive a first tissue of the patient therein, the layered composite comprising:
a structural layer configured to support the first tissue after implantation into the anatomy of the patient; and
an absorbent layer joined to the structural layer and comprising a material configured to retain platelet rich plasma in a desired position within the anatomy of the patient; and
a flange portion comprising the layered composite extending radially from the tubular portion to a circumferential edge and defining a third edge contiguous with the first edge and a fourth edge continuous with the second edge to define a second portion of the installation slit, wherein the flange portion defines a first surface and a second surface, and wherein at least one of the first surface and the second surface is configured to abut a second tissue of the patient.

2-4. (canceled)

5. The surgical implant of claim 1, wherein the absorbent layer includes at least one of a coagulation cofactor, thrombin, and calcium dichloride.

6. (canceled)

7. The surgical implant of claim 1, further including a second flange portion extending radially from the tubular portion to a second circumferential edge and defining a fifth edge contiguous with the first edge and a sixth edge continuous with the second edge to define a third portion of the installation slit.

8. (canceled)

9. The surgical implant of claim 1, wherein the surgical implant includes at least two flange portions extending radially from the tubular portion.

10. The surgical implant of claim 5, wherein each of the at least two flange portions are positioned at a point that is between the distal end and the proximal end of the tubular portion.

11. The surgical implant of claim 1, wherein at least one of the flange portion and the tubular portion extend about an arc that is within a range from about 180-degrees to above 360-degrees, and wherein at least one of the flange portion and the tubular portion is configured to retain its shape when cut.

12. A method of preventing or treating tissue prolapse disorders in a human patient, the method comprising:
providing a surgical implant that includes:
a tubular portion comprising a layered composite extending axially from a distal end to a proximal end and circumferentially from a first edge to a second edge, wherein the first edge and the second edge define a first portion of an installation slit extending along a longitudinal length of the tubular portion, wherein the tubular portion defines a lumen extending from the distal end to the proximal end and configured to receive a first tissue of the patient therein, the layered composite comprising:
a structural layer configured to support the first tissue after implantation into the anatomy of the patient; and
an absorbent layer joined to the structural layer and comprising a material configured to retain platelet rich plasma in a desired position within the anatomy of the patient; and
a flange portion comprising the layered composite extending radially from the tubular portion to a circumferential edge and defining a third edge contiguous with the first edge and a fourth edge contiguous with the second edge to define a second portion of the installation slit, wherein the flange portion defines a first surface and a second surface, and wherein at least one of the first surface and the second surface is configured to abut a second tissue of the patient; positioning the first tissue of the patient within the lumen of the tubular portion; and positioning the flange portion of the implant against the second tissue of the patient.

13. The method of claim 12, wherein the tissue prolapse disorder is a herniated esophageal tissue and wherein positioning the first tissue of the patient within the lumen of the tubular portion includes positioning esophageal tissue within the lumen.

14. The method of claim 13, wherein a lower esophageal sphincter is positioned within the lumen of the tubular portion.

15. The method of claim 13, wherein positioning the flange portion of the implant against the second tissue of the patient includes abutting the flange portion against tissue of a diaphragm or a stomach.

16. The method of claim 12, wherein the tissue prolapse disorder is a prolapsed rectum, wherein positioning the first tissue of the patient within the lumen of the tubular portion includes positioning colon tissue within the lumen, and wherein the second tissue comprises at least a portion of the pelvic floor.

17. The method of claim 16, wherein rectal tissue is positioned within the lumen of the tubular portion.

18. The method of claim 12, wherein the tissue prolapse disorder is a prolapsed uterus, wherein positioning the first tissue of the patient within the lumen of the tubular portion includes positioning uterine tissue within the lumen, and wherein the second tissue comprises at least a portion of the pelvic floor.

19. The method of claim 12, further including applying a therapeutic substance to the implant, wherein the therapeutic substance is platelet rich plasma.

20-34. (canceled)

35. A surgical implant for treating tissue prolapse or hiatal hernia disorders in a patient, the implant comprising a composite defining:
a tubular portion extending axially from a distal end to a proximal end and circumferentially from a first edge to a second edge, wherein the first edge and the second edge define a first portion of an installation gap extending along a longitudinal length of the tubular portion, wherein the tubular portion defines a lumen extending from the distal end to the proximal end configured to receive a first tissue of the patient therein; and
a flange portion extending radially from the tubular portion to a circumferential edge and defining a third edge and a fourth edge each extending radially from the tubular portion to define a second portion of the installation gap, wherein the flange portion defines a surface configured to abut a second tissue of the patient, wherein the first tissue and the second tissue are separate anatomical structures,
wherein the composite comprises:
a structural portion configured to support at least one of the first tissue and the second tissue after implantation into the anatomy of a patient; and
an absorptive portion comprising a material configured to allow a platelet-rich plasma to pass through at least a portion of the absorptive portion and retain the platelet-rich plasma against at least one of the first tissue and the second tissue to promote the formation of at least one of neoplastic, connective tissue and collagen.

36. The surgical implant of claim 35, wherein at least one of the structural portion and the absorptive portion comprise a woven fabric defining the tubular portion and the flange portion.

37. The surgical implant of claim 35, wherein at least one of the structural portion and the absorptive portion defines a plurality of pleats, and wherein the other of the structural layer and the absorbative layer is joined to at least a portion of the pleats to fix the pleats.

38. The surgical implant of claim 37, wherein the layered composite further comprises a third layer is joined to at least a portion of the pleats to fix the pleats.

39. The surgical implant of claim 35, wherein the layered composite further comprises a radio-opaque material.

40. The surgical implant of claim 35, wherein the absorptive portion comprises a plurality of pores configured to retain the platelet-rich plasma, and wherein a size of each pore of the plurality of pores is less than about 3 millimeters.
